(19) United States
(12) Patent Application Publication
(10) Pub. No.: US 2005/0222260 A1
(43) Pub. Date: Oct. 6, 2005

(54) (2S)-2-amino-a-{[2-(ethanimidoylamino) ethyl]thio}butanoic acid, a nitric oxide synthase inhibitor, in stabilized pharmaceutical dosage forms

(76) Inventors: Stuart James Broughton, Ware (GB); Rajinder Kumar Gharu, Ware (GB); Mark Yuon Tuck Leow, Ware (GB); Philip John Neale, Ware (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/517,801

(22) PCT Filed: Jun. 19, 2003
(86) PCT No.: PCT/EP03/06465

§ 371 Date: Dec. 10, 2004

(51) Int. Cl. A61K 31/195

(52) U.S. Cl. 514/562

(57) ABSTRACT

Pharmaceutical compositions comprising (2S)-2-amino-4-{[2-(ethanimidoylamino) ethyl]thio}butanoic acid, a pharmaceutically acceptable bulking agent and one or more antioxidants or chelating agents are described.

The present invention relates to novel pharmaceutical formulations of (2S)-2-amino-4-[2-(ethanimidoylamino)ethyl]thio}butanoic acid, to processes for their manufacture, and to their use in therapy.

Nitric oxide is the endogenous stimulator of the soluble guanylate cyclase enzyme and is involved in a number of biological actions. Excess nitric oxide production is also thought to be involved in a number of conditions, including septic shock and many inflammatory diseases. The biochemical synthesis of nitric oxide from L-arginine is catalysed by the enzyme NO synthase. Many inhibitors of NO synthase have been described and proposed for therapeutic use.

More recently, it has been an object in this field to provide NOsynthase inhibitors displaying selectivity for either inducible NO synthase (iNOS) or neuronal NO synthase (nNOS) over endothelial NO synthase (eNOS).

Thus WO98/30537 describes selective NO synthase inhibitors of formula 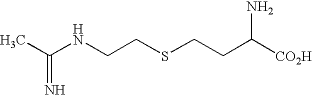
or a salt, solvate, or physiologically functional derivative thereof.

Co-pending patent application number PCT/GB01/05596 (published as WO02/50021) describes a phosphate salt of (2S)-2-amino4-{[2-(ethanimidoylamino)ethyl]thio}butanoic acid, and solvates thereof, which has advantageous properties, in particular, being considerably less hygroscopic than, for example, the hydrochloride salts exemplified in WO98/30537.

We have found that (2S)-2-amino4-{[2-(ethanimidoylamino)ethyl]thio}butanoic acid, for example, in the form of its compound with phosphoric acid, exhibits incompatability with standard pharmaceutical excipients conventionally used in the preparation of solid oral dosage forms such as tablets. This incompatibility results in increased degradation of the (2S)-2-amino-4-{[2-(ethanimidoylamino)ethyl]thio}butanoic acid and reduced stability of the formulations. We have further found that these problems can be overcome by the addition to solid oral dosage forms comprising (2S)-2-amino4-([2-(ethanimidoylamino)ethyl]thio}butanoic acid and conventional pharmaceutical excipients of an antioxidant and/or a chelating agent.

According to the present invention there is provided a pharmaceutical composition comprising (2S)-2-amino-4-{[2-(ethanimidoylamino)ethyl]thio}butanoic acid, a pharmaceutically acceptable bulking agent and one or more antioxidants or chelating gents.

For use in accordance with the present invention, the (2S)-2-amino4-{[2-(ethanimidoylamino)ethyl]thio}butanoic acid will preferably be in the form of its compound with phosphoric acid, preferably in hydrated form. More preferably each molecule (2S)-2-amino-4-{[2-(ethanimidoylamino)ethyl]thio)butanoic acid is associated with at least one molecule of water, such as 1, 2 or 3 water molecules, especially 1 or 3 water molecules, preferably 1 water molecule. Suitably, the (2S)-2-amino-4-[2-(ethanimidoylamino)ethyl]thio}butanoic acid comprises from about 0.1 to about 5% by weight, preferably about 0.25 to about 2.5%, more preferably about 0.5 to about 1%, such as about 0.6%, based on the dry weight of the dosage form.

It will be appreciated by those skilled in the art that phosphoric acid exists in more than one form. The preferred form for use in the context of the present invention isorthophosphoric acid (H₃PO₄).

Pharmaceutically acceptable bulking agents are inert substances that provide bulk and dilute the active ingredient when used in the compositions of the present invention. Suitable pharmaceutically acceptable bulking agents will be well known to the person skilled in the art and a non-limiting list of examples includes microcrystalline cellulose (available as, for example, Avicel™), croscarmellose sodium, crospovidone, starch, calcium carboxymethylcellulose, silicified microcrystalline cellulose, magnesium oxide, tragacanth, dibasic calcium phosphate dihydrate, anhydrous dibasic calcium phosphate (available as, for example, Emcompress™), tribasic calcium phosphate, calcium carbonate, magnesium carbonate, calcium sulfate, cellulose acetate, powdered cellulose, kaolin, polymethacrylates and talc. The pharmaceutically acceptable bulking agents may be used alone or in combination with each other. Preferred is Avicel™, such as, for example, Avicel™ PH101, Avicel™ PH102, Avicel™ PH112, Avicel™ PH113 and Avicel™ PH200, starch, and mixtures thereof. Particularly preferred pharmaceutically acceptable bulking agents for use in the present invention are Avicel™ PH101, starch 1500 and mixtures thereof.

Suitably, the pharmaceutically acceptable bulking agents comprise from about 80 to about 99.5% by weight, preferably about 90 to about 99%, more preferably about 95 to about 98.3%, based on the dry weight of the dosage form.

Suitable antioxidants and chelating agents for use in the compositions of the present invention will be pharmaceutically acceptable and include, for example, organic acids such as ethylenediaminetetraacetic acid (EDTA), ascorbic acid, malic acid, citric acid and fumaric acid, and salts and esters thereof such as, for example, sodium ascorbate and propyl gallate, salts of inorganic acids such as, for example, sodium metabisulfite, alcohols such as, for example, maltol and alpha tocopherol and aromatic compounds such as, for example, butylated hydroxyanisole and butylated hydroxytoluene. Preferred are EDTA, malic acid and ascorbic acid and mixtures thereof, especially EDTA, malic acid and mixtures thereof.

Suitably, the antioxidant, chelating agent, or mixture thereof comprises from about 0.005 to about 5% by weight, preferably about 0.01 to about 2.5%, more preferably about 0.05 to about 2%, such as about 0.1%, based on the dry weight of the dosage form.

Thus, in a further embodiment of the present invention, there is provided a pharmaceutical composition as hereinbefore described wherein the (2S)-2-amino-4-{[2-(ethanimidoylamino)ethyqthio}butanoic acid comprises from about 0.1 to about 5% by weight, preferably about 0.25 to about 2.5%, more preferably about 0.5 to 1%, especially about 0.6%, the pharmaceutically acceptable bulking agent comprises from about 80 to about 99.5% by weight, preferably about 90 to about 99%, more preferably about 95 to about 98.3%, and the antioxidant, chelating agent, or mixture thereof comprises from about 0.005 to about 5% by weight, preferably about 0.01 to about 2.5%, more preferably about 0.05 to about 2%, especially about 0.1%, based on the dry weight of the dosage form.

In a further embodiment of the present invention, there is provided a pharmaceutical composition as hereinbefore described wherein the (2S)-2-amino-4-[2-(ethanimidoylamino)ethyl]thio}butanoic acid is in the form of its compound with phosphoric acid, preferably in hydrated form, more preferably as the monohydrate or the trihydrate, the pharmaceutically acceptable bulking agent comprises Avice™, preferably Avicel™ PH101, Avicel™ PH102, Avicel™ PH112, Avicel™ PH113 or Avicel™ PH200, or starch, or, more preferably, a mixture of Avicel™ and starch, the antioxidant, chelating agent, or mixture thereof comprises EDTA, malic acid or ascorbic acid, or a mixture of EDTA and malic acid or a mixture of EDTA and ascorbic acid. Particularly preferred are compositions comprising (2S)-2-amino-4-[2-(ethanimidoylamino)ethyl]thio}butanoic acid compound with phosphoric acid, 1:1, monohydrate, Avicel™ PH101, optionally starch 1500, and EDTA, malic acid or a mixture of EDTA and malic acid.

In addition to the ingredients described above, the pharmaceutical composition of the present invention may further comprise pharmaceutically acceptable excipients such as binding agents (e.g. pregelatinised starch, polyvinylpyrrolidone, hydroxypropylmethyl-cellulose) and lubricants (e.g. stearic acid, silicon dioxide, magnesium stearate, talc, sodium benzoate and hydrogenated vegetable oil).

Many drug substances have an inherently bitter taste. The unpleasant taste that is sometimes associated with oral administration of a composition comprising (2S)-2-amino-4-{[2-(ethanimidoylamino)ethyl]thio}butanoic acid may be substantially eliminated by the use of a film coat on the solid core. The solid core comprises (2S)-2-amino-4-{[2-(ethanimidoylamino)ethyl]thio}butanoic acid.

Accordingly, in one embodiment, the present invention provides a pharmaceutical composition as hereinbefore described in the form of a tablet which is film-coated.

The film coating may suitably comprise a polymer. Suitable polymers will be well known to the person skilled in the art and a non-limiting list of examples includes cellulose ethers, for example, hydroxypropylmethylcellulose, hydroxypropylcellulose or methylcellulose, and copolymers of methacrylic acid and methyl methacrylate.

The total film coating solids are generally applied to the solid dosage form, for example the tablet core, in an amount of from about 0.5 to 10% by weight, preferably about 1 to about 4%, more preferably about 2 to about 3%, based on the dry weight of the dosage form.

The film coating may additionally comprise any pharmaceutically acceptable colourants or opacifiers including water soluble dyes, aluminium lakes of water soluble dyes and inorganic pigments such as titanium dioxide and iron oxide.

The film coating may also contain one or more plasticising agents conventionally used in polymeric film coatings, for example, polyethylene glycol, propylene glycol,dibutyl sebecate, mineral oil, sesame oil, diethyl phthalate and triacetin. Proprietary film coating materials, such as Opaspray and Opadry, obtainable from Colorcon Ltd., UK, may be used.

The taste of oral compositions may also be improved by the use of flavouring and/or sweetening agents. Suitable flavouring agents will be well known to the person skilled in the art and a non-limiting list of examples includes lemon, orange, grapefruit, vanilla, caramel, butterscotch, hazelnut or mint flavouring. Suitable sweetening agents for use in the compositions of the invention will be well known to the person skilled in the art and a non-limiting list of examples includes sucrose, saccharin, cyclamic acid and alkali or alkali earth metal salts thereof, mannitol, acesulfame-K, stevioside, thaumatin and aspartame. The flavouring and/or sweetening agents may be used alone or in combination with each other.

As mentioned above, the compound of formula (I) is an inhibitor of NO synthase.

In a further aspect, the present invention provides a pharmaceutical composition for oral administration as hereinbefore described for use in the treatment of clinical conditions for which an inhibitor of NO synthase is indicated, in particular an inhibitor of iNOS. Such conditions include inflammatory conditions, shock states, immune disorders, disorders of gastrointestinal motility and diseases of the central nervous system including migraine and metabolic disorders including dyslipidemia.

By shock states is meant those resulting from overproduction of NO, such as septic shock, haemorrhagic shock, traumatic shock, or shock caused by fulminant hepatic failure or by therapy with cytokines such as TNF, IL-1 and IL-2 or therapy with cytokine-inducing agents, for example 5,6-dimethylxanthenone acetic acid.

Examples of inflammatory conditions and immune disorders include those of the joint, particularly arthritis (e.g. rheumatoid arthritis, osteoarthritis, prosthetic joint failure), or the gastrointestinal tract (e.g. ulcerative colitis, Crohn's disease, and other inflammatory bowel diseases, gastritis and mucosal inflammation resulting from infection, the enteropathy provoked by non-steroidal anti-inflammatory drugs), of the airways (e.g. adult respiratory distress syndrome, asthma, cystic fibrosis, upper respiratory tract inflammatory disease (e.g. rhinitis such as allergic rhinitis) or chronic obstructive pulmonary disease), of the heart (e.g. myocarditis), of nervous tissue (e.g. multiple sclerosis), of the pancreas (e.g. diabetes melitus and complications thereof), of the kidney (e.g. glomerulonephritis), of the skin (e.g. dermatitis, psoriasis, eczema, urticaria), of the eye (e.g. glaucoma) as well as of transplanted organs (e.g. rejection) and multi-organ diseases (e.g. systemic lupus erythematosis) and inflammatory sequelae of viral or bacterial infections.

Furthermore, there is evidence for overproduction of NO by INOS in atherosclerosis and following hypoxic or ischaemic insults (with or without reperfusion), for example in the brain or in ischaemic heart disease.

Disorders of gastrointestinal motility include ileus, for example post-operative ileus and ileus during sepsis.

By diseases of the central nervous system is meant those for which overproduction of NO is implicated, for example migraine, psychosis, anxiety, schizophrenia, sleep disorders, cerebral ischaemia, CNS trauma, epilepsy, multiple sclerosis, AIDS dementia, chronic neurodegenerative disease (e.g. Lewy Body Dementia, Huntington's disease, Parkinson's disease, or Alzheimer's disease) and acute and chronic pain, and conditions in which non-adrenergic non-cholinergic nerves may be implicated such as priapism, obesity and hyperphagia.

Examples of acute pain include musculoskeletal pain, post operative pain and surgical pain. Examples of chronic pain include chronic inflammatory pain (e.g. rheumatoid arthritis and osteoarthritis), neuropathic pain (e.g. post herpetic neuralgia, diabetic neuropathies associated with diabetes, trigeminal neuralgia, pain associated with functional bowel disorders, e.g. irritable bowel syndrome, non cardiac chest pain and sympathetically maintained pain) and pain associated with cancer and fibromyalgia.

Overproduction of NO is implicated in metabolic disorders through its effect on lipoprotein lipase activity causing hypertriglyceridemia. Selective inhibitors of iNOS will be useful in metabolic conditions where NO over-production is implicated, such as dyslipidaemia.

Furthermore, inhibition of NO synthase may be of advantage in preventing the lymphocyte loss associated with HIV infection, in increasing the radiosensitivity of tumours during radiotherapy and in reducing tumour growth, tumour progression, angiogenesis, and metastasis.

It will be appreciated that reference to treatment is intended to includeprophylaxis as well as the alleviation of established symptoms.

Accordingly, the present invention provides a method for the treatment of a clinical condition in a mammal, such as a human, for which an inhibitor of nitric oxidesynthase, for example, an iNOS inhibitor is indicated, which comprises oral administration of a pharmaceutical composition as hereinbefore described. In particular, the present invention provides a method for the treatment of an inflammatory and/or immune disorder, such as arthritis, allergic rhinitis, COPD or asthma. In a further preferred aspect the present invention provides a method for the prophylaxis or treatment of a clinical condition selected from pain, migraine, ileus and irritable bowel syndrome.

According to a further aspect of the invention, there is provided the use of a pharmaceutical composition for oral administration as hereinbefore described in the manufacture of a medicament for treating conditions for which an inhibitor of nitric oxide synthase, for example, an iNOS inhibitor is indicated. In particular, the present invention provides the use of a pharmaceutical composition for oral administration as hereinbefore described in the manufacture of a medicament for treating an inflammatory and/or immune disorder, such as arthritis, allergic rhinitis, COPD or asthma, pain, migraine, ileus or irritable bowel syndrome.

In the alternative, there is also provided a pharmaceutical composition for oral administration as hereinbefore described for use in medical therapy, particularly for use in the prophylaxis or treatment of a clinical condition in a mammal, such as a human, for which an inhibitor of nitric oxide synthase, for example an iNOS inhibitor, is indicated. In particular, there is provided a pharmaceutical composition for oral administration as hereinbefore described for the treatment of an inflammatory and/or immune disorder, such as arthritis, allergicrhinitis, COPD or asthma, pain, migraine, ileus or irritable bowel syndrome.

The amount of (2S)-2-amino-4[2-(ethanimidoylamino)ethyl]thio}butanoic acid which is required to achieve a therapeutic effect will, of course, vary with the route of administration, the subject under treatment, and the particular disorder or disease being treated. Typically a dose of from 0.001 to 200 mg/kg per day, preferably 0.01 to 20 mg/kg per day may be employed. The dose range for adult humans is generally from 0.1 mg to 10 g/day and preferably 1 mg to 1 g/day.

For the preparation of compositions according to the invention, the active ingredient, the antioxidant and/or chelating agent and optional additional excipients such as lubricants, etc. may be blended with the pharmaceutically acceptable bulking agent. If desired, one or more of the components of this powder blend may be granulated. A process of, for example, dry granulation, wet granulation or solvent granulation may optionally be used. Dry granulation is generally preferred. Tablets may be prepared, for example, by compression of the powder blend. Capsules may be prepared, for example, by filling of the powder blend into suitable capsule shells.

The solid dosage form may be film-coated using a suspension comprising a suitable polymer in a suitable solvent. The preferred solvent for the film coating components is purified water but various classes of organic solvents commonly used in this art such as alcohols, ketones, ethers and chlorinated hydrocarbons, for example ethanol, acetone, dichloromethane and the like, may also be used. The solvent does not appear in the final product.

Herein, the term “active ingredient” means (2S)-2-amino-4-(2-(ethanimidoylamino)ethyl]thio}butanoic acid and pharmaceutically acceptable salts and solvates thereof.

The invention is illustrated by the folowing non-limiting examples wherein Compound A is (2S)-2-amino4-([2-(ethanimidoylamino)ethyl]thio} butanoic acid, compound with phosphoric acid, monohydrate.

### EXAMPLE 1

### Direct Compression Formulation for (2S)-2-amino4{2-(ethanimidoylamino)ethyl]thio}butanoic acid, compound with phosphoric acid, monohydrate (Compound A)

Formula 1

| Component | Percent w/w | |
|---|---|---|
| Compound A | 0.62 | |
| EDTA | 0.1 | |
| Avicel PH101 | 98.28 | |
| Silicon Dioxide | 0.5 | |
| Magnesium Stearate | 0.5 | |
| Total Tablet Weight | 250 | mg |

Dry Granulation Method

The components other than magnesium stearate are weighed from bulk containers, sieved using either hand held screens or a Ytron-Quadro Comil 197AS and deposited into a stainless-steel blending container. The components are blended for up to 30 minutes using a suitable blender, such as an Apex MPX, Fordertechnik FT or Glatt GPCG. The magnesium stearate is then added to the mixture and blending is continued for approximately 2 minutes. The lubricated blend is then compressed using a suitable rotary tablet press, such as a Unipress B/D. The tablets are coated using a film coater such as an Accelacota 10 or Glatt GC500.

Wet Granulation Method

The Compound A and EDTA (and/or, as appropriate, an antioxidant) are dissolved in a volume of water (approx. 2.5 to 3.0 litres) and sprayed onto bulk excipient particles (i.e. Avicel), granulated and dried using a mixer granulator, such as a PMA10. The dry granules are blended using a suitable blender, such as an Apex MPX or Fordertechnik FT with further Avicel, silicon dioxide and magnesium stearate. The lubricated blend is then compressed using a suitable rotary press, typically a Unipress B/D press.

The tablets are coated using a film coater such as an Accelacota 10 or Glatt GC500.

Formulations according to the following examples were similarly prepared:

### EXAMPLE 2

| Component | Percent w/w | |
|---|---|---|
| Compound A | 0.62 | |
| Ascorbic acid | 0.1 | |
| Avicel PH101 | 98.28 | |
| Silicon Dioxide | 0.5 | |
| Magnesium Stearate | 0.5 | |
| Total Tablet Weight | 250 | mg |

### EXAMPLE 3

| Component | Percent w/w | |
|---|---|---|
| Compound A | 0.62 | |
| Malic acid | 0.1 | |
| Avicel PH101 | 98.28 | |
| Silicon Dioxide | 0.5 | |
| Magnesium Stearate | 0.5 | |
| Total Tablet Weight | 250 | mg |

### EXAMPLE 4

| Component | Percent w/w | |
|---|---|---|
| Compound A | 0.62 | |
| EDTA | 0.1 | |
| Ascorbic acid | 0.1 | |
| Avicel PH101 | 98.18 | |
| Silicon Dioxide | 0.5 | |
| Magnesium Stearate | 0.5 | |
| Total Tablet Weight | 250 | mg |

### EXAMPLE 5

| Component | Percent w/w | |
|---|---|---|
| Compound A | 0.62 | |
| EDTA | 0.1 | |
| Avicel PH101 | 58.28 | |
| Starch 1500 | 40.0 | |
| Silicon Dioxide | 0.5 | |
| Magnesium Stearate | 0.5 | |
| Total Tablet Weight | 250 | mg |

### EXAMPLE 6

| Component | Percent w/w | |
|---|---|---|
| Compound A | 0.62 | |
| Ascorbic acid | 0.1 | |
| Avicel PH101 | 58.28 | |
| Starch 1500 | 40.0 | |
| Silicon Dioxide | 0.5 | |
| Magnesium Stearate | 0.5 | |
| Total Tablet Weight | 250 | mg |

### EXAMPLE 7

| Component | Percent w/w | |
|---|---|---|
| Compound A | 0.62 | |
| Malic acid | 0.1 | |
| Avicel PH101 | 58.28 | |
| Starch 1500 | 40.0 | |
| Silicon Dioxide | 0.5 | |
| Magnesium Stearate | 0.5 | |
| Total Tablet Weight | 250 | mg |

### EXAMPLE 8

| Component | Percent w/w | |
|---|---|---|
| Compound A | 0.62 | |
| EDTA | 0.1 | |
| Ascorbic acid | 0.1 | |
| Avicel PH101 | 58.18 | |
| Starch 1500 | 40.0 | |
| Silicon Dioxide | 0.5 | |
| Magnesium Stearate | 0.5 | |
| Total Tablet Weight | 250 | mg |

### EXAMPLE 9

| Component | Percent w/w | |
|---|---|---|
| Compound A | 0.62 | |
| EDTA | 0.1 | |
| Malic acid | 0.1 | |
| Avicel PH101 | 58.18 | |
| Starch 1500 | 40.0 | |
| Silicon Dioxide | 0.5 | |
| Magnesium Stearate | 0.5 | |
| Total Tablet Weight | 250 | mg |

Stabilibty Results

1 mg tablet formulations corresponding to Examples 1 -8 (each tablet contains 1 mg of (2S)-2-amino-4-{2-(ethanimidoylamino)ethyl]thio}butanoic acid) were prepared by dry granulation, essentially as described in Example 1 but mixing by hand using a pestle and mortar.

The impurity content of the formulations thus prepared was measured (initial time point) by high pressure liquid chromatography (HPLC). Samples of the formulations were stored at 40° C., 75% relative humidity and 50° C., ambient humidity for three months. The impurity content (greatest impurity and total impurities) was measured at 14 days, 1 month and 3 month time points. The HPLC system used was an Aglient 1100 HPLC system. Typical chromatographic conditions were:
Analytical column: Prodigy ODS 3 5 μm 150×4.6 mm
Mobile Phase: 87:13 Aq Component (0.02M pot.dihydrogen orthophosphate/0.01M)
Flow rate: 1.5 ml/min
Temp: 40 deg C.
Detection: UV @205 nm
Inj Vol: 10 μL
Approx. run time: 25 min

Moisture content of Compound A was determined using a Mitsubushi CA06 Moisture meter connected to a Mitsubushi VA06 Vaporiser
N2 flow: 200 ml/min
Stirrer speed: 5
Delay time: 30 secs
Sensitivity: 0.1 μg sec-1
Temp: 135 deg C.
Vapouriser cycle: 1-1-1
Back range time: 1 min
Samp boat removal time: 1 min
Samp boat cooling time: 1 min
Reagents Anode: Aquamicron AKX or AX (150 ml) Cathode: Aquamicron CXU (2 ampoules)

The results are shown in Table 1. The greatest chemical stability was seen for the formulations corresponding to Examples 3, 5 and 7.

TABLE 1

| | Impurity Content | | | | | | | | | | | | | |
|---|---|---|---|---|---|---|---|---|---|---|---|---|---|---|
| | Greatest | | | | | | | Total | | | | | | |
| | | DY14 | | MN1 | | MN3 | | | DY14 | | MN1 | | MN3 | |
| Formula- | | 40/ | 50/ | 40/ | 50/ | 40/ | 50/ | | 40/ | 50/ | 40/ | 50/ | 40/ | 50/ |
| tion | INT | 75 | Amb | 75 | Amb | 75 | Amb | INT | 75 | Amb | 75 | Amb | 75 | Amb |
| Ex 1 | 0.16 | 0.17 | 0.16 | 0.16 | 0.21 | 1.92 | 0.25 | 0.2 | 0.3 | 0.4 | 0.4 | 0.5 | 4.7 | 0.7 |
| Ex 2 | 0.48 | 0.21 | 0.44 | 0.25 | 0.40 | 4.31 | 0.32 | 1.0 | 0.6 | 1.0 | 0.8 | 1.0 | 8.8 | 1.0 |
| Ex 3 | 0.19 | 0.17 | 0.17 | 0.21 | 0.16 | 0.95 | 0.25 | 0.3 | 0.3 | 0.5 | 0.5 | 0.5 | 2.2 | 0.7 |
| Ex 4 | 0.17 | 0.17 | 0.16 | 0.18 | 0.16 | 3.65 | 0.26 | 0.3 | 0.4 | 0.4 | 0.5 | 0.5 | 8.1 | 0.8 |
| Ex 5 | 0.17 | 0.16 | 0.16 | 0.42 | 0.16 | 1.03 | 0.17 | 0.3 | 0.3 | 0.4 | 1.5 | 0.4 | 2.8 | 0.6 |
| Ex 6 | 0.75 | 0.17 | 0.64 | 0.42 | 0.2 | 3.63 | 0.34 | 1.5 | 0.4 | 1.3 | 1.1 | 0.6 | 7.4 | 0.9 |
| Ex 7 | 0.19 | 0.17 | 0.21 | 0.17 | 0.19 | 0.61 | 0.28 | 0.4 | 0.4 | 0.6 | 0.4 | 0.6 | 1.9 | 0.8 |
| Ex 8 | 0.16 | 0.17 | 0.18 | 0.19 | 0.22 | 1.92 | 0.34 | 0.4 | 0.4 | 0.5 | 0.5 | 0.6 | 4.2 | 0.9 |

INT = initial time point
DY14 = day 14
MN1 = 1 month
MN3 = 3 months

The application of which this description and claims forms part may be used as a basis for priority in respect of any subsequent application. The claims of such subsequent application may be directed to any novel feature or combination of features described herein. This may take the form of product, composition, process or use claims and may include, by way of example and without limitation, one or more of the following claims:

1. A pharmaceutical composition comprising (2S)-2-amino-4-{[2-(ethanimidoylamino)ethyl]thio}butanoic acid, a pharmaceutically acceptable bulking agent and one or more antioxidants or chelating agents.

2. The pharmaceutical composition as claimed in claim 1 wherein the (2S)-2-amino-4-{[2-(ethanimidoylamino)ethyl]thio}butanoic acid is in the form of its (1:1) compound with phosphoric acid, or a solvate thereof.

3. The pharmaceutical composition as claimed in claim 2 wherein the solvate is a hydrate.

4. The pharmaceutical composition as claimed in claim 3 wherein the hydrate is the monohydrate.

5. The pharmaceutical composition as claimed in claim 3 wherein the hydrate is the trihydrate.

6. The pharmaceutical composition as claimed in claim 1 wherein the (2S)-2-amino-4-{[2-(ethanimidoylamino)ethyl]thio}butanoic acid comprises from about 0.1 to about 5% by weight, the pharmaceutically acceptable bulking agent comprises from about 80 to about 99.5% by weight, and the antioxidant, chelating agent, or mixture thereof comprises from about 0.005 to about 5% by weight, based on the dry weight.

7. The pharmaceutical composition as claimed in claim 1 wherein the antioxidants or chelating agents are selected from the group comprising EDTA, malic acid, ascorbic acid and mixtures thereof.

8. The pharmaceutical composition as claimed in claim 1 wherein the pharmaceutically acceptable bulking agent comprises microcrystalline cellulose, starch or a mixture thereof.

9. A method for the treatment or prophylaxis of a clinical condition in a mammal, for which an inhibitor of nitric oxide synthase is indicated, which comprises administration of a pharmaceutical composition as claimed in claim 1.

10. The method as claimed in claim 9 wherein the clinical condition is selected from arthritis, asthma, rhinitis, chronic obstructive pulmonary disease, ileus, migraine, pain and irritable bowel syndrome.

11. (canceled)

12. (canceled)

13. (canceled)

14. (canceled)

15. (canceled)

16. The method as claimed in claim 9 wherein said mammal is a human.
